(19) United States
(12) Patent Application Publication
(10) Pub. No.: US 2024/0285178 A1
(43) Pub. Date: Aug. 29, 2024

(54) PULSE MONITORING DEVICE AND SYSTEM INCLUDING THE SAME

(71) Applicant: National Tsing Hua University, Hsinchu City (TW)

(72) Inventors: Wei-Chih Wang, Sammamish, WA (US); Chun-Cheng Liu, Hsinchu City (TW); Fiona Marie Wang, Seattle, WA (US); Hao-Min Cheng, Hsinchu City (TW); Chen-Huan Chen, Hsinchu City (TW)

(21) Appl. No.: 18/173,626

(22) Filed: Feb. 23, 2023

(51) Int. Cl. A61B 5/021; A61B 5/00

(52) U.S. Cl. CPC A61B 5/02108; A61B 5/002; A61B 5/6824; A61B 5/6831; A61B 5/7207; A61B 5/742; A61B 5/02427; A61B 5/02438; A61B 2562/166; A61B 2562/223; A61B 2562/228

(57) ABSTRACT

Provided is an optical sensor for monitoring pulse waveform and blood pressure of a subject. The optical sensor may be manufactured with compact structure, low profile, low cost, and exhibits benefits of disposability, easy to apply, immunity to electro-magnetic interference, high sensitivity, having minimal affect towards sense of touch, maintains patient safety, and supportive of accurate real-time measurements for the clinician. Therefore, pulse waveform and blood pressures of a subject may be faithfully monitored continuously throughout day and night, so as to provide abundant prognostic information while avoiding interference in normal daily activity of the subject. Also provided is a system utilizing the optical sensors for monitoring pulse waveform and blood pressure of a subject.

### TECHNICAL FIELD

The present disclosure relates to clinical examination techniques, and more particularly to a wearable optical sensor and a system for monitoring pulse waveform and blood pressure of a subject.

### DESCRIPTION OF RELATED ART

The primary cause of death and fatal illness in human is cardiovascular disease (CVD). Researchers have found that pulse waveform patterns may accurately correlate with an increased risk of developing CVD. Pulse is one of the most critical physiological signals of human that comes directly from heart to the blood vessel system, which is useful for providing abundant and reliable information about cardiovascular system. For example, pulse reflections will occur at different level of blood vessels as pulses are being transmitted. Further, conditions such as resistance of blood flow, elastic of vessel wall and blood viscosity may influence waveform patterns of pulses. Moreover, pathological changes may also affect pulse waveform patterns in aspect of strength, reflection, or frequency thereof.

Common instruments for non-invasively measuring pulse include a cuff-based blood pressure monitoring device such as ambulatory blood pressure monitoring (ABPM) device. The ABPM device is a pressure cuff that enables monitoring of a patient's level change in blood pressure over a short-term basis (within 24 hours). Thus, the ABPM device is capable of monitoring morning surge (MS) in blood pressure, which may be related to some cardiovascular complications in the morning (e.g. early morning hypertension, hemorrhagic stroke, myocardial infarction, or, stroke). However, such cuff-based blood pressure monitoring device usually causes apparent discomfort during measurement while the patient is asleep, thus disturbing the patient's sleep. Also, poor sleep quality may reduce nocturnal blood pressure fall and thus produce offset to the patient's true sleep-trough blood pressure, which may reduce the accuracy of the measurement. Moreover, current cuff-based blood pressure monitoring device also poses other disadvantages such as cost for reimbursement, limited availability in private practice, susceptibility to electromagnetic interference, low sensitivity, and discomfort in patients.

To counter the lack of accuracy and autonomy in conventional instruments for measuring pulses (as mentioned above), various arterial pulse wave sensors have been widely reported recently due to easy application for outpatients and tentative diagnosis. Among these existing products, the optical biosensor is an option for meeting the needs of immunity to electromagnetic interference and higher sensitivity. However, the current optical biosensors are usually still large, cumbersome, and incompatible to operate along with normal clinical cardiovascular exam.

Therefore, it is an unmet need to develop a non-invasive pulse monitoring device with blood pressure measurement and system that can resolve the aforementioned problems in the art.

### SUMMARY

In view of the foregoing, the present disclosure provides an optical sensor for monitoring pulse waveform and blood pressure of a subject, comprising a cover, an input waveguide, and one or more detection waveguides.

In at least one embodiment of the present disclosure, the cover has a chamber formed therein and has an input port and an output port formed on the outer surface of the cover. In some embodiments of the present disclosure, the cover is configured to attach to the skin of the subject.

In at least one embodiment of the present disclosure, the input waveguide has a first end and a second end and configured to guide a light from the first end to the second end. In some embodiments of the present disclosure, the first end is configured to receive the light from a light source, and the second end is configured to transmit the light into the chamber of the cover. In some embodiments of the present disclosure, the first end is coupled to the light source directly or by an optical fiber, and the second end of the input fiber is disposed in the chamber through the input port of the cover.

In at least one embodiment of the present disclosure, the optical sensor comprises a first detection waveguide having a third end and a fourth end and configured to guide the light from the third end to the fourth end. In some embodiments of the present disclosure, the third end is configured to receive the light transmitted from the second end of the input waveguide, and the fourth end is configured to transmit the light to a first detector. In some embodiments of the present disclosure, the third end is disposed in the chamber of the cover through the output port thereof, and the forth end is coupled to the first detector directly or by an optical fiber. In some embodiments of the present disclosure, the second end of the input waveguide is not in contact with the third end of the first detection waveguide, and the second end of the input waveguide is configured to move relatively to the third end of the first detection waveguide. In some embodiments of the present disclosure, the first detection waveguide comprises four micro waveguides forming a quadrant detector configured in a quadrant configuration or a cross configuration.

In at least one embodiment of the present disclosure, the optical sensor comprises a second detection waveguide having a fifth end and a sixth end and configured to guide the light from the fifth end to the sixth end. In some embodiments of the present disclosure, the fifth end is configured to receive the light transmitted from the second end of the input waveguide, and the sixth end is configured to transmit the light to a second detector. In some embodiments of the present disclosure, the fifth end is disposed in the chamber of the cover through the output port thereof, and the sixth end is coupled to the second detector directly or by an optical fiber. In some embodiments of the present disclosure, the second end of the input waveguide is not in contact with the fifth end of the second detection waveguide, and the second end of the input waveguide is configured to move relatively to the fifth end of the second detection waveguide.

In at least one embodiment of the present disclosure, the chamber is sealed with a soft membrane configured to contact with or attach to the skin of the subject. In some embodiment of the present disclosure, the soft membrane is configured to attach the cover to the skin of the subject. In some embodiment of the present disclosure, a first signal regarding the pulse waveform and blood pressure of the subject is produced by the first detector based on an attenuation in a light coupling of the light between the input waveguide and the first detection waveguide, and the attenuation in the light coupling occurs when: (1) the second end of the input waveguide moves upward relatively to the third end of the first detection waveguide since the membrane constricts to cause high pressure within the chamber in response to constriction of the skin during systole of an artery underneath the skin: or (2) the second end of the input waveguide moves downward relatively to the third end of the first detection waveguide since the membrane constricts to cause low pressure within the chamber in response to expansion of the skin during diastole of an artery underneath the skin.

In some embodiment of the present disclosure, the optical sensor comprises a cantilever having a free end coupled to the second end of the input waveguide in the chamber. In some embodiments of the present disclosure, the cantilever is a plastic deforming plate.

In at least one embodiment of the present disclosure, the optical sensor comprises an enclosure having a free end coupled to the second end of the input waveguide in the chamber. In some embodiments of the present disclosure, the optical sensor comprises a cylindrical applicator penetrating through the cover, and the cylindrical applicator has a first side in contact with the cantilever and a second side opposite the first side. In some embodiments of the present disclosure, the optical sensor comprises a bump disposed on the second side of the corrugated applicator to improve contact of the corrugated applicator between the skin of the subject and the cantilever. In some embodiments of the present disclosure, a first signal regarding the pulse waveform and blood pressure of the subject is produced by the first detector based on an attenuation in a light coupling of the light between the input waveguide and the first detection waveguide, and the attenuation in the light coupling occurs if the cylindrical applicator drives the second end of the input waveguide coupled with the free end of the cantilever to move upward relatively to the third end of the first detection waveguide as the skin of the subject constricts during systole of an artery underneath the skin. In some embodiments of the present disclosure, a second signal regarding the pulse waveform and blood pressure of the subject is produced by the second detector based on an attenuation in a light coupling of the light between the input waveguide and the second detection waveguide, and the attenuation in the light coupling occurs if the cylindrical applicator drives the second end of the input waveguide coupled with the free end of cantilever to move upward relatively to the fifth end of the second detection waveguide as the skin of the subject constricts during systole of an artery underneath the skin.

In at least one embodiment of the present disclosure, the third end of the first detection waveguide is adjacent and parallel to the fifth end of the second detection waveguide. In some embodiments of the present disclosure, when pulse of the subject is not detected yet, the second end of the input waveguide is disposed along the middle line of the third end of the first detection waveguide and the fifth end of the first detection waveguide.

The present disclosure also provides an optical sensor for monitoring pulse waveform and blood pressure of a subject, comprising: a first plate having a first engaging side, a second plate having a second engaging side facing the first engaging side, and an optical fiber sandwiched between the first engaging side of the first plate and the second engaging side of the second plate.

In at least one embodiment of the present disclosure, the first engaging side and the second engaging side have corrugated teeth structure formed thereon for engaging the optical fiber. In some embodiments of the present disclosure, the corrugated teeth are spaced from each other with a pitch, and the optical fiber exhibits periodic bending at the pitch of the corrugated teeth. In some embodiments of the present disclosure, the optical fiber is configured to guide a light from a light source to a detector, and attenuation of the light in the optical fiber is proportional to amount of the periodic bending of the optical fiber. In some embodiments of the present disclosure, a signal regarding the pulse waveform and blood pressure of the subject is produced by the detector by detecting a light loss during the attenuation of the light related to a force applied to the optical sensor.

In the present disclosure, the optical sensor may be manufactured with compact structure, low profile, low cost, and exhibits benefits of disposability, easy to apply, immunity to electro-magnetic interference, high sensitivity, having minimal affect towards sense of touch, maintains patient safety, and supportive of accurate real-time measurements for the clinician.

The present disclosure also provides a system for monitoring pulse waveform and blood pressure of a subject, comprising: a transmitter comprising a light source and a detector and configured to collect and deliver signal regarding the pulse waveform produced by the detector: the optical sensor of the present disclosure which is coupled to the transmitter by a connector and configured to guide a light from the light source to the detector; and a processing device configured to process the signal delivered by the transmitter.

In at least one embodiment of the present disclosure, the transmitter comprises a housing having an interface slot for accommodating the connector. In some embodiments of the present disclosure, the connecter has a taper structure for allowing insertion into the interface slot or relieving strain of the input waveguide or the first detection waveguide.

In at least one embodiment of the present disclosure, the transmitter comprises an optical printed circuit board disposed in the housing and configured for carrying the light source and the detector, powering the light source, and/or receiving the signal from the detector, a radio printed circuit board disposed in the housing and configured for carrying a Bluetooth radio and/or sending the signal to the processing device, and a printed circuit board carrying a micro controller unit for managing functionality of the transmitter. In some embodiments of the present disclosure, the printed circuit board carrying the micro controller further carries a control interface for programing and testing of functionality of the transmitter, and wherein the control interface comprises a 7-segment display, a voltage regulator, a header, a touch sensor, or any combination thereof.

In at least one embodiment of the present disclosure, the connector comprises a male part for receiving the input waveguide and the first detection waveguide, and a female part for connecting the input waveguide and the first detection waveguide to the transmitter in accordance with disposition of the light source and the detector. In some embodiments of the present disclosure, the male part is couple to the female part. In some embodiments of the present disclosure, the male part is magnetically coupled to the female part. In some embodiments of the present disclosure, the male part has a first set of magnets, and the female part has a second set of magnets, wherein the first set of magnets and the second set of magnets corresponds in numbers and location to allow self-aligned coupling between the male part and the female part. In some embodiments of the present disclosure, the transmitter is configured to not power on unless the first set of magnets and the second set of magnets are aligned in place.

In at least one embodiment of the present disclosure, the processing device is configured with a data acquisition (DAQ) system for analyzing the signal delivered by the transmitter and a graphical user interface (GUI) for displaying the pulse waveform and blood pressure according to the signal.

In at least one embodiment of the present disclosure, the transmitter further comprising a wristband, wherein the housing has a ring structure configured to hold a wristband, and the optical sensor is embedded in the wristband near a radial artery of the subject.

The present disclosure also provides a method for monitoring pulse waveform and blood pressure of a subject, comprising: providing the optical sensor of the present disclosure; measuring the pulse waveform and blood pressure of the subject by the optical sensor to generate a pulse waveform and blood pressure signal; recognizing motion signal generated by the subject; filtering the motion signal from the pulse waveform and blood pressure signal; and reconstructing an accurate pulse waveform and blood pressure signal.

In at least one embodiment of the present disclosure, the motion signal is generated by the motion such as subject's breathing, hand, arm and knee movement, or the subject engaging in walking or running

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The present disclosure can be more fully understood by reading the following descriptions of the embodiments, with reference made to the accompanying drawings.

FIG. 1 is a schematic diagram of a system for monitoring pulse waveform of a subject in accordance with embodiments of the present disclosure.

FIGS. 2A-2B are schematic diagrams illustrating a stereo view and a cross-sectional view of a fiber-optic bend loss sensor in accordance with embodiments of the present disclosure, respectively.

FIG. 3 is a schematic diagram illustrating light attenuation within a fiber-optic bend loss sensor in accordance with embodiments of the present disclosure.

FIGS. 4A-4C are function graphs illustrating design factors for a fiber-optic bend loss sensor in accordance with embodiments of the present disclosure.

FIG. 5 is a schematic diagram illustrating sensing methodologies of a discontinuous fiber optic sensor in accordance with embodiments of the present disclosure.

FIGS. 6A-6B are cross-sectional diagrams illustrating an embodiment of a discontinuous fiber optic sensor in accordance with embodiments of the present disclosure.

FIG. 7 is a cross-sectional diagram illustrating another embodiment of a discontinuous fiber optic sensor that can measure 1D motion in accordance with embodiments of the present disclosure.

FIG. 7A is a cross-sectional diagram illustrating another embodiment of a discontinuous fiber optic sensor that can measure 2D motion in accordance with embodiments of the present disclosure.

FIGS. 7B-1 and 7B-2 are enlarged schematic diagrams illustrating another embodiment of the detection fibers that can measure 2D motion in accordance with embodiments of the present disclosure.

FIGS. 8A-8B are cross-sectional diagrams illustrating a further embodiment of a discontinuous fiber optic sensor in accordance with embodiments of the present disclosure.

FIG. 9 is a cross-sectional diagram illustrating designing factors for a discontinuous waveguide optical sensor in accordance with embodiments of the present disclosure.

FIG. 10 is a circuit diagram illustrating designing factors for a discontinuous fiber optic sensor in accordance with embodiments of the present disclosure:

FIG. 11 is a cross-sectional diagram illustrating a transmitter in accordance with embodiments of the present disclosure; and

FIGS. 12A-12B are schematic diagrams illustrating an exploded view and assembly view of a connector in accordance with embodiments of the present disclosure, respectively.

FIG. 13A and FIG. 13B are function graphs illustrating the corresponding X displacement and Y displacement spectrum from the quadrant sensor of the present disclosure.

FIG. 14A is a function graph illustrating the example of head, hand, foot movement detected by the sensor of the present disclosure.

FIG. 14B is a function graph illustrating the example of respiration and heart beat detected by the sensor of the present disclosure.

FIG. 15A is a function graph illustrating examples of knee bending as a function of frequencies with joint angles.

FIG. 15B is a function graph illustrating examples of knee bending as a function of frequencies with and joint moments.

### DETAILED DESCRIPTION OF THE EMBODIMENTS

The following embodiments are used for illustrating the present disclosure. A person having ordinary skill in the art can easily conceive the other advantages and effects of the present disclosure, based on the disclosure of the specification. The present disclosure can also be implemented or applied as described in different examples. It is possible to modify or alter the examples for carrying out this disclosure without contravening its scope, for different aspects and applications.

It is further noted that, as used in this specification, the singular forms “a,” “an,” and “the” include plural referents unless expressly and unequivocally limited to one referent. The term “or” is used interchangeably with the term “and/or” unless the context clearly indicates otherwise.

As used herein, the term “waveguide” refers to an element or structure for guiding a light, including but not limited to a fiber, a rib, a slab, a strip, a wire, a rod, a tubes, a buried channel, or a slot. In at least one embodiment of the present disclosure, the waveguide may be flexible, which means the waveguide may be bendable without destroying the function or structure thereof. In at least one embodiment of the present disclosure, the waveguide is an optical fiber, a rectangular rib, or a slab.

As used herein, the term “light” refers to electromagnetic radiation of whatever wavelength, for example, infrared light (e.g., about 750 nm to 1 mm), visible light (e.g., about 400 nm to 700 nm), ultraviolet, telecommunications (e.g., about 1260 nm to 1675 nm). Accordingly, the term “light,” “optical,” and the similar terms thereof used herein are not limited to the visible range.

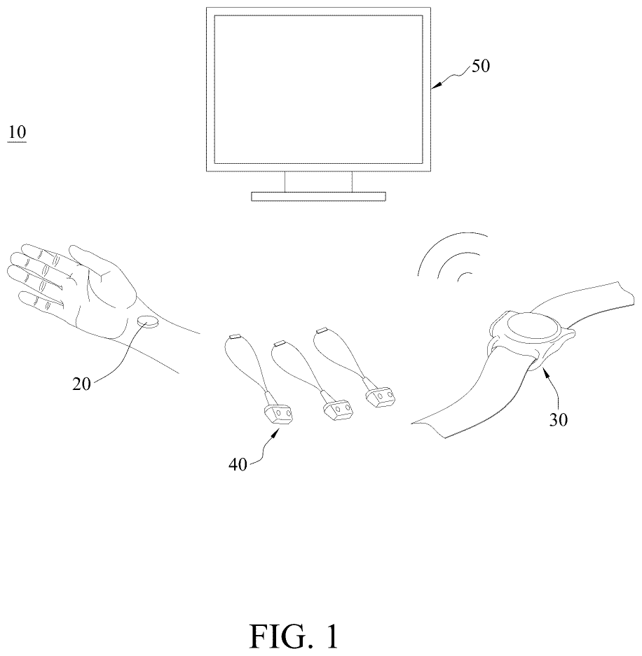

Referring to FIG. 1, in which a system 10 for continuous monitoring of pulse waveform with blood pressure of a subject (e.g. a patient) is provided. Specifically, the system 10 comprises one or more optical sensors 20, a transmitter 30, one or more connectors 40, and a processing device 50. The aforesaid components can operate independently or respectively. Further, these components may be in electrical communication with each other through wired or wireless manner such as Bluetooth communication. Moreover, the system 10 can either designed with a standalone single optical sensor 20 or multiple optical sensors 20 to allow monitoring of user-defined location(s).

In the embodiments described herein, the optical sensors 20 are patch sensors configured to attach to the skin of the subject and continuously monitor pulse waveform and blood pressure (or with blood pressure) of the subject.

As for a single sensor design, a single optical sensor 20 may be placed at wrist of the subject to monitor the subject's pulse waveforms (e.g. for peripheral artery blood pressure monitoring). The optical sensor 20 may also be placed on, but not limited to, carotid artery, ventral tips of fingers of the subject, palm of the subject, or other suitable locations on demands.

As for a multiple sensor design, multiple optical sensors 20 may be placed on different locations of the subject for monitoring different heart conditions simultaneously. For example, optical sensors 20 may be placed at ankle or arm of the subject to allow measurement of ankle-brachial index, which suggests subject's arteriosclerosis condition indicating geriatric or chronic diseases. The multiple optical sensors 20 may also be placed on other body parts of the subject along its central and peripheral arteries: however, the present disclosure is not limited thereto. More specifically, the multiple optical sensors 20 may be placed on the body of the subject corresponding to its carotid artery (central artery), brachial artery, radial artery (peripheral artery), dorsalis pedis artery, or the like, so as to collect various pulse waveforms and blood pressures from the subject. The detailed configuration of the optical sensor(s) 20 will be described hereinafter.

In the embodiments described herein, the connectors 40 corresponds in quantity toward the transmitter 30. The optical sensors 20 may be coupled with the transmitter 30 through the connectors 40. The detailed configurations of the connectors 40 will be further described hereinafter.

In the embodiments described herein, the transmitter 30 is configured as a base station where multiple optical sensors 20 (or only one optical sensor 20) are interfaced so as to collect and deliver (to the processing device 50) signals regarding the pulse waveforms and blood pressures measured by respective optical sensors 20. In some embodiments, the transmitter 30 is realized as a wearable device such as a watch unit.

In other embodiments, the transmitter 30 may also be realized in other forms such as, but not limited to, a patch device, a neckwear device, a wristband device or the like. Further, there may be multiple transmitters 30 for a single subject to handle optical sensors 20 on various body parts of the subject simultaneously, of which the present disclosure is also not limited thereto. The detailed configuration of the transmitter 30 will be further will be further described hereinafter.

In the embodiments described herein, the processing device 50 is configured to process and display the signals collected and delivered by the transmitter 30. In some embodiments, the processing device 50 may be realized as any computing apparatus or service such as, but not limited to, a desk top computer, a laptop computer, a tablet, a smart phone device, a cloud computing service, or the like. The detailed configuration of the processing device 50 will be further described hereinafter.

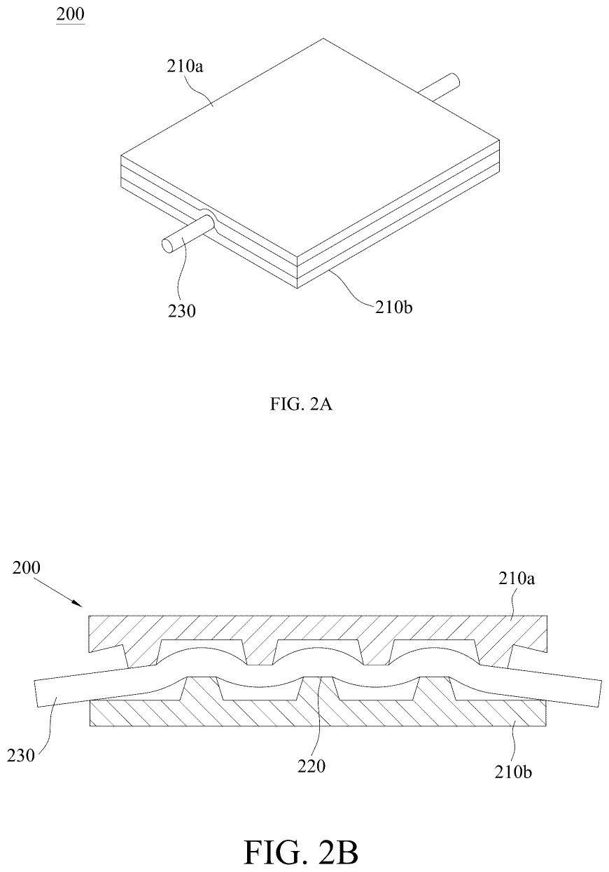

FIGS. 2A-2B show a first embodiment of the optical sensors 20. In this first embodiment, the optical sensors 20 are realized as fiber-optic bend loss sensors 200, which comprise: a pair of plates 210a and 210b having their engaging sides facing each other, a series of corrugated teeth 220 formed on the engaging sides of the plates 210a and 210b, and an optical fiber 230. The optical fiber 230 is sandwiched between the pair of plates 210a and 210b, while the optical fiber 230 is engaged by the series of corrugated teeth 220 when the pair of plates 210a and 210b are compressed together by outer force so as to produce periodic bending in the optical fiber 230.

In some embodiments, the pair of plates 210a and 210b are thin polymer plates that forms a patch when compressed together and attached to skin of the subject, and the optical fiber 230 is a multimode optical fiber. Further, the shape of the patch formed by plates 210a and 210b described herein is a square or a rectangle. However, the shape of the patch may be any kind of shape based on design requirements.

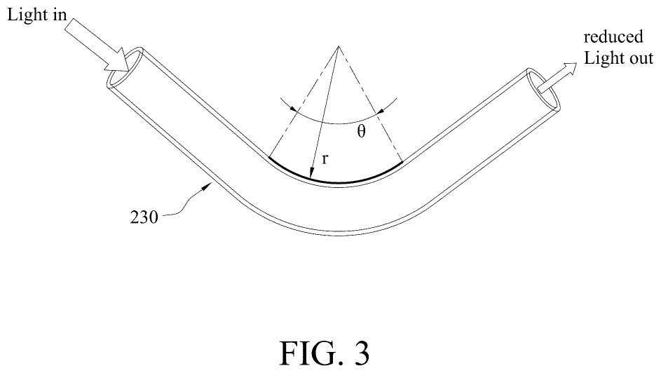

In further embodiments, the semi-rigid configuration of pair of plates 210a and 210b (i.e. having configurations of the corrugated teeth 220) are engineered to induce periodic bending in the optical fiber 230 at a pitch, which attenuates light (e.g. a light traveling from a light source to a detector) passing through the optical fiber 230. That is, the attenuation of light is proportional to the amount of bending the optical fiber 230 experiences at said pitch. In this way, the force (e.g. the pulse propagated through skin) applied may directly related to the light lost (e.g. optical bend loss) detected by the detector during attenuation (as illustrated in FIG. 3), enabling the fiber-optic bend loss sensors 200 to be electronically inert and highly sensitive to manual forces applied by the hands.

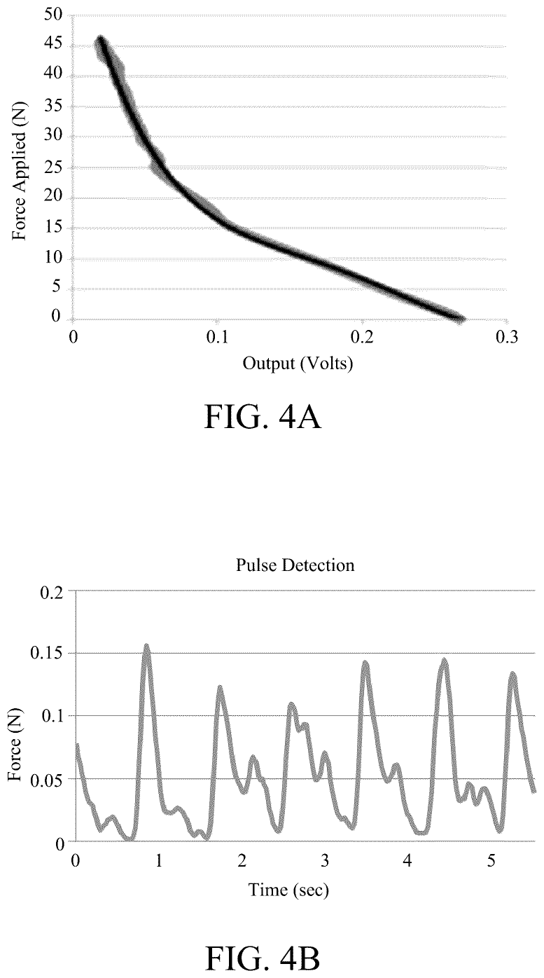

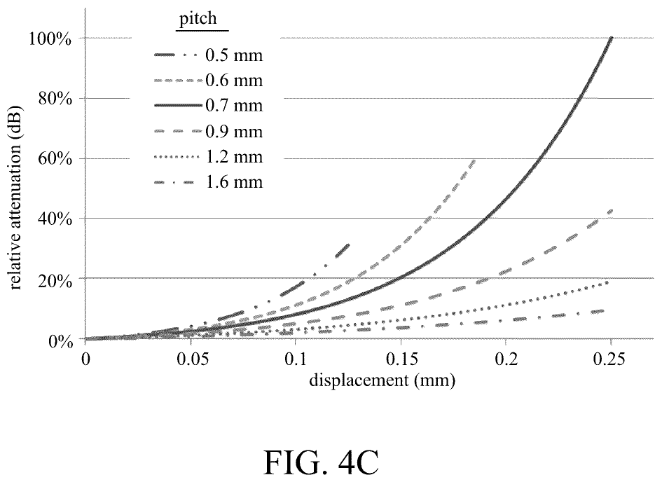

In preferred embodiments, the fiber-optic bend loss sensors 200 should possess a non-linear sensitivity profile that provides high sensitivity at low forces and large range at higher forces due to its novel combination of geometry and optical bend loss characteristics. FIG. 4A illustrates a desired performance of the fiber-optic bend loss sensors 200 when being tested under static and dynamic calibration processes such as changing of temperature in the climate the subject is located and changing of tissue stiffness where the fiber-optic bend loss sensors 200 are attached to. In particular, FIG. 4A illustrates a function plot regarding applied force (in units of N) and output (in units of Volts) when a fiber-optic bend loss sensor 200 being tested is applied with force range from 0.03 N to 80 N in resolution of 0.01 N, where the calibration curve (the black curved line) derived from actually-detected data (the gray distribution of dots) should display in monotonically increasing manner with hysteresis of maximum error of 4.0 N. Therefore, with the performance illustrated in FIG. 4A, the fiber-optic bend loss sensor 200 may perform dynamic pulse detection in high sensitivity and fidelity up to a maximum loading rate of 100 N/second, as shown in FIG. 4B.

The total power loss due to bending can be simplified to an exponential function of bending radius and angle:

$${L}_{total} = α {e}^{β θ - γ r} \qquad ( 1 )$$

where L is the total light loss in dB, θ is the total angle about which the optical fiber 230 is bent (360° for a full revolution around a mandrel), r is the radius of curvature of the bent fiber, and α, β, and γ are constants dependent on various conditions such as fiber type and wavelength. Each of these constants is determined experimentally. We can then conclude that the sensor 200 that produces bending in the optical fiber 230 around a profile with large radius r and small curvature θ will yield maximal attenuation.

The correlation between displacement and bending loss of the optical fiber 230 can be geometrically calculated. To obtain bending loss as a function of force applied to the sensor only requires the relationship between force and displacement which can be computationally generated with finite element modeling.

The equations relating the radius of curvature r and bending angle θ as a function of force were then used to generate a theoretical light loss formula based on bending formulas calculated and verified.

When bending the optical fiber 230 between a series of cylindrical teeth 220 spaced closely enough to approximate successive radial bends in the fiber, bending (and consequently sensitivity) depends on the pitch of the teeth (p), the radius of curvature (Rₜ) of the teeth 230 and the height (Hₜ) of the teeth 230. The latter two parameters affect the displacement range attainable by the sensor 200 because of mechanical obstruction. Loss has been empirically measured and shown by the micro bend loss equation above. The displacement (d) of the sensor 200 has been shown to be linear with respect to force (F).

The effect of displacement on bending can be geometrically calculated as demonstrated below. In this calculation, the optical fiber 230 is assumed to contact both teeth 220 of the pair of plates 210a and 210b tangentially throughout loading and will be unbent when unloaded (d=0). The bent fiber can be approximated as two arcs tangent to each other and equal in dimension. The average radius of curvature of the optical fiber 230 is approximated through the axis of the optical fiber 230.

Because the fiber bends in two equal portions, segment b is one half of sensor displacement d. Similarly, segment a is one half of the pitch (p) because it is the midpoint between the teeth. Therefore, by the Pythagorean Theorem

$${c}^{2} = {a}^{2} + {b}^{2} = {( p / 2 )}^{2} + {( d / 2 )}^{2} \qquad ( 2 )$$

By law of cosines

$$cos θ = \frac{2 {r}^{2} - {c}^{2}}{2 {r}^{2}} \qquad ( 3 )$$

Which, after substitution for c² and reducing, becomes

$$cos θ = 1 - \frac{{p}^{2} + {d}^{2}}{8 {r}^{2}} \qquad ( 4 )$$

To obtain a system of 2 equations to solve for θ and r, we can define the trigonometric relationship

$$cos θ = \frac{r - d / 2}{r} \qquad ( 5 )$$

Solving the system gives

$$r = \frac{{p}^{2} - {d}^{2}}{4 d} \qquad ( 6 )$$

and

$$θ = {cos}^{- 1} ( 1 - \frac{2 {d}^{2}}{{p}^{2} - {d}^{2}} ) \qquad ( 7 )$$

The formula for r can be approximated as a reciprocal function (r=0.2/d) when d is less than 0.25 mm and the formula for θ can be approximated as a linear function (θ=0.13*d) with respect to displacement in the same region.

These mathematical models validate the finite element analysis performed with the same geometry. These formulas can also be inserted into the simplified bendloss formula to estimate the relationship between attenuation and displacement. The relationship between attenuation and displacement is dependent on the coefficients of each factor (angle and radius) in equation 1 and thus the radius and angle components cannot be easily combined analytically to obtain a loss formula. Other theoretical formulae have been demonstrated to calculate these correction factors.

The relative attenuation due to decreasing the radius of curvature by loading is found to be

$${L}_{r} = α {e}^{- γ r} = α {e}^{- γ * \frac{{p}^{2} - {d}^{2}}{4 d}} \qquad ( 8 )$$

As tooth pitch is increases, attenuation decreases until the mechanical limit is reached.

Relative attenuation due to increasing the angle of bending is given by

$${L}_{θ} = α {e}^{β θ} = α {e}^{β * {cos}^{- 1} ( 1 - \frac{2 {d}^{2}}{{p}^{2} - {d}^{2}} )} \qquad ( 9 )$$

Attenuation is most severe at lower pitches, but the mechanical limit of the sensor is also demonstrated here. Sensitivity to bending is most profound with the smallest pitch (0.5 mm) but has the highest ultimate range when the teeth are spaced 0.7 mm apart. Sensitivity to bending is again lowest at low displacements.

The total loss can be combined from Equations 8 and 9:

$${L}_{T} = α {e}^{β θ - γ r} = α {e}^{β * {cos}^{- 1} ( 1 - \frac{2 {d}^{2}}{{p}^{2} - {d}^{2}} ) - γ * \frac{{p}^{2} - {d}^{2}}{4 d}} \qquad ( 10 )$$

In further embodiments, in order to make the fiber-optic bend loss sensors 200 more sensitive to pulses of the subject (such as the performances shown in FIGS. 4A), the design of the corrugated teeth 220 and the corresponding periodic bending in the optical fiber 230 may be modified based on a micro bend loss equation shown below:

$${L}_{T} = α {e}^{βθ - γ r} = α {e}^{β * {cos}^{- 1} ( 1 - \frac{2 {d}^{2}}{{p}^{2} + {d}^{2}} ) - γ * \frac{{p}^{2} + {d}^{2}}{4 d}}$$

where L represents the total light loss in measures of dB, θ represents the total angle that the optical fiber 230 is bent, r represents the radius of curvature of the bent optical fiber 230, p represents the pitch between adjacent corrugated teeth 220 encountered by the optical fiber 230, and α, β, and γ are constants dependent on various conditions such as fiber type of the optical fiber 230 and wavelength of the light entering the optical fiber 230. Moreover, FIG. 4C illustrates a function plot derived from the above equation that describes the relative attenuation of light in the optical fiber 230 with regard to the pitch design between adjacent corrugated teeth 220, where the pitch of 0.7 mm represents the maximum displacement between adjacent corrugated teeth 220 that achieves best attenuation of light. Thus, the best sensitivity of the fiber-optic bend loss sensors 200 can be achieved.

In some embodiments, the slightly curve contours of the top and bottom surfaces reinforce the applicator plates (the plates 210a or 210b) so that the optical fiber 230 can bend normal when a load is applied, and the sensor 200 is also made naturally to respond to a force that is added to a point since the light attenuation is proportional to the total amount of bend occur in the entire optical fiber 230 and independent to the individual bends in teeth 220.

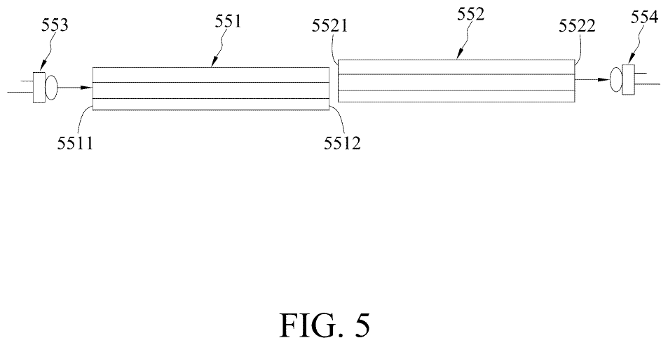

In other embodiments of this disclosure, the optical sensors 20 are embodied as discontinuous fiber optic sensors. FIG. 5 shows a basic concept of the discontinuous fiber optic sensors, where the design is similar to concepts used in a microphone design showed a sub μPa pressure detection that may help achieve three to four times of sensitivity than conventional pulse waveform detection products. In particular, the design of the discontinuous fiber optic sensors focuses on transverse alignment between the input fiber 551 and the detection fiber 552. The input fiber 551 has a first end 5511 and a second end 5512, and the detection fiber 552 has a third end 5521 and a fourth end 5522. For example, when misalignment between the input fiber 551 and the detection fiber 552 occurs due to blood pressure pulse-induced deformation of a skin above an artery, the light from the light source 553 (e.g. a LED, a NIR-LED, or other types of emitters) attenuates at the light coupling between the input fiber 551 and the detection fiber 552. Therefore, the attenuation in the light coupling causes a power falling detected on fiber detector 554, hence allows magnitude of the blood pressure to be measured.

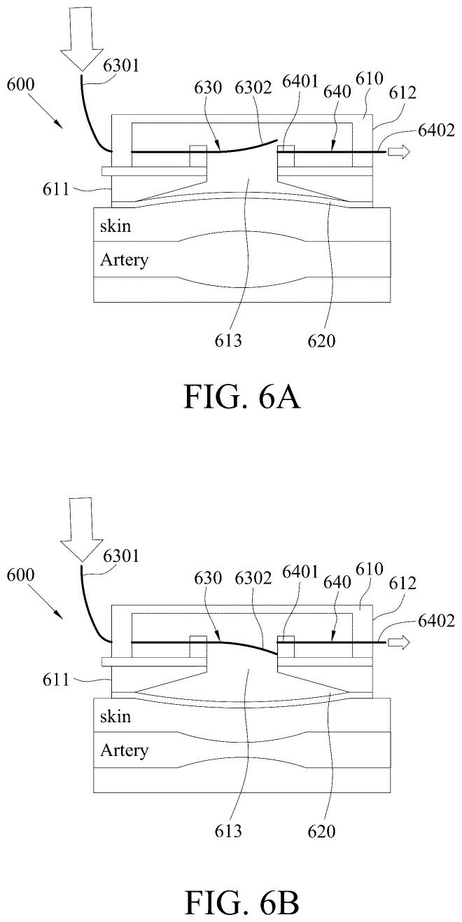

FIGS. 6A and 6B show a second embodiment of optical sensors 20 which is embodied as discontinuous fiber optic sensors 600. The discontinuous fiber optic sensors 600 comprise: a cover 610, an input fiber 630 and a detection fiber 640. The cover 610 is of opening design that has an input side 611 with an input port, an output side 612 with an output port and opposed to the input side 611, and a chamber 613 is formed in the coved 610 between the input side 611 and the output side 612. The input fiber 630 is realized as a cantilever fiber that has a first end 6301 connect to a light source (not shown) and a second end 6302 disposed in the chamber 613 through the input port of the input side 611, and the input fiber 630 is configured to guide a light from the light source to the first end 6301 and to the second end 6302. The detection fiber 640 has a third end 6302 disposed in the chamber 613 and configured to receive the light transmitted form the second end 6302 of the input fiber and has a fourth end 6402 connected to the detector (not shown), and the detection fiber 640 is configured to guide a light from the third end 6401 to the fourth end 6402 and to the detector. An opening in the chamber 613 is sealed by a membrane 620. In some embodiments, the membrane 620 is used to attach the discontinuous fiber optic sensor 600 to the subject's skin.

As shown from FIG. 6A to 6B, the second end 6302 of the input fiber 630 may be move relatively to the third end of the detection fiber 640 with pulse of the subject. The discontinuous fiber optic sensors 600 monitor pulse waveform and blood pressure of the subject through change in transverse alignment (as illustrated in FIG. 5) between input fiber 630 and the detection fiber 640 in response to high and/or low pressure occurred in the chamber 613 during systole and diastole of the artery under the skin (e.g. blood pressure induced vibration) where the discontinuous fiber optic sensors 600 is attached. To be exact, during systole of the artery, the skin (along with the membrane 620) of the subject constricts and produces high pressure in the chamber 613, hence lifting (with respect to the skin surface) the input fiber 630 to cause misalignment between the input fiber 630 and the detection fiber 640 (FIG. 6A), and a pulse waveform correlated to systole of the artery may be detected. On the other hand, during diastole of the artery, the skin (along with the membrane 620) of the subject expands and produces low pressure in the chamber 613, hence bowing (with respect to the skin surface) the input fiber 630 to cause misalignment between the input fiber 630 and the detection fiber 640 (FIG. 6B), and a pulse waveform correlated to diastole of the artery may be detected.

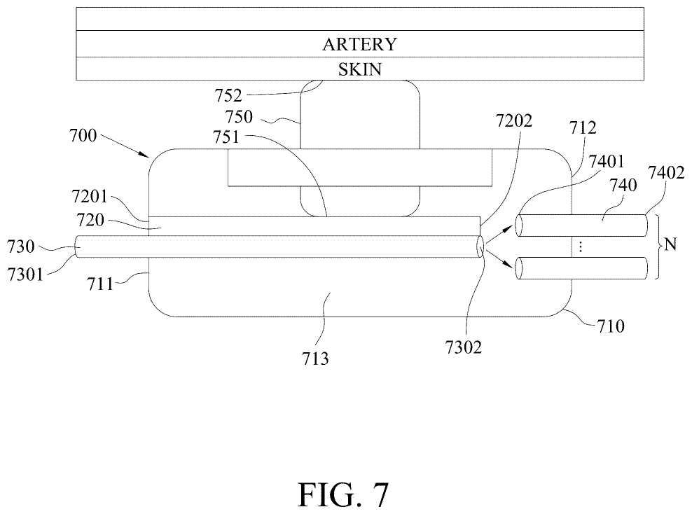

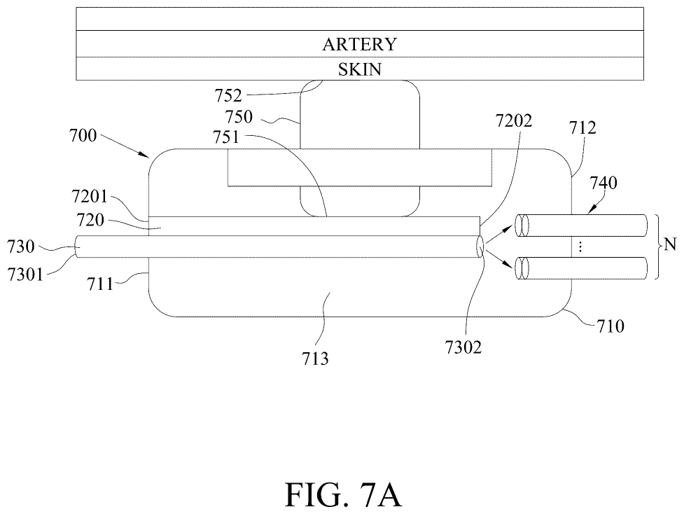

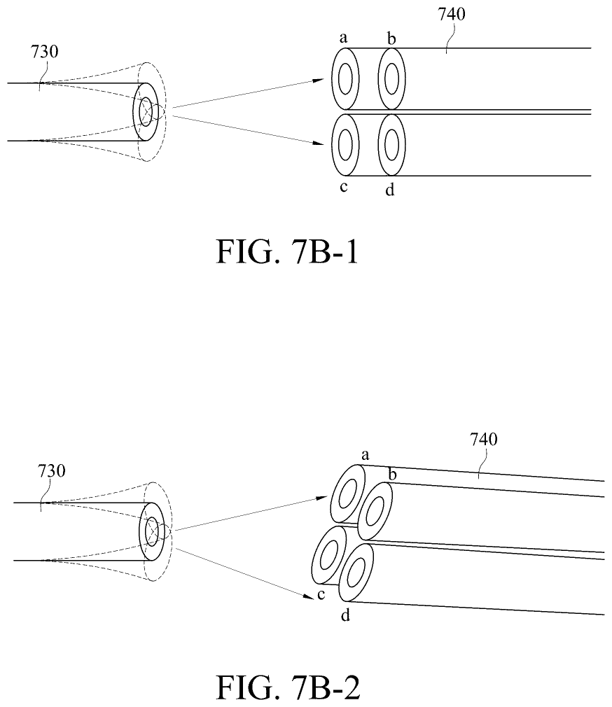

FIG. 7 shows a third embodiment of the optical sensors 20 which is embodied as discontinuous fiber optic sensors 700. The discontinuous fiber optic sensors 700 comprise: a cover 710, a cantilever 720, an input fiber 730 carried by the cantilever 720 and a plurality of detection fibers 740 adjacent and parallel to each other. The cover 710 is of sealed design that has an input side 711 with an input port, an output side 712 opposed to the input side 711 and having a plurality of output port formed thereon in accordance with the number of detection fibers 740, and a chamber 713 formed in the cover 710 and housing the cantilever 720, the input fiber 730 and the plurality of detection fibers 740. The cantilever 720 has a fixed end 7201 and a free end 7202 disposed in the chamber 713. The input fiber 730 has a first end 7301 connect to a light source (not shown) and a second end 7302 disposed in the chamber 713 through the input port of the input side 711, and the input fibers 730 are configured to guide a light from the light source to the first end 7301 and to the second end 7302. Each of the detection fibers 740 has a third end 7302 disposed in the chamber 713 and configured to receive the light transmitted form the second end 7302 of the input fiber 730 and has a fourth end 7402 connected to one of the detectors (not show), and the detection fibers 740 is configured to guide a light from the third end 7401 to the fourth end 7402 and to the detectors. The free end 7202 of the cantilever 720 is coupled to the second end of the input fiber 730 and can move relatively to the third end 7401 of the detection fibers 740 with the pulse of the subject. In some embodiments, the cantilever 720 is a plastic deforming plate.

The discontinuous fiber optic sensors 700 further comprise a cylindrical applicator 750 arranged through the cover 710 on a skin-contacting side. The cylindrical applicator 750 has a first side 751 contacting the cantilever 720 and a second side 752 opposed to the first side 751 that enables attachment with the subject's skin.

In some embodiments, the cylindrical applicator 750 exhibits a similar function as the membrane 620 shown in FIG. 6, which is to responsively react to systole and diastole of the artery of the subject. Therefore, with the cylindrical applicator 750 being in contact with the cantilever 720 and the skin of the subject simultaneously, and with the input fiber 730 being attached to the cantilever 720, the input fiber 730 can be displaced relative to a movement of the attached cantilever 720 (via the cylindrical applicator 750) caused by diastole and/or systole of the artery underneath the skin (e.g. blood pressure induced vibration).

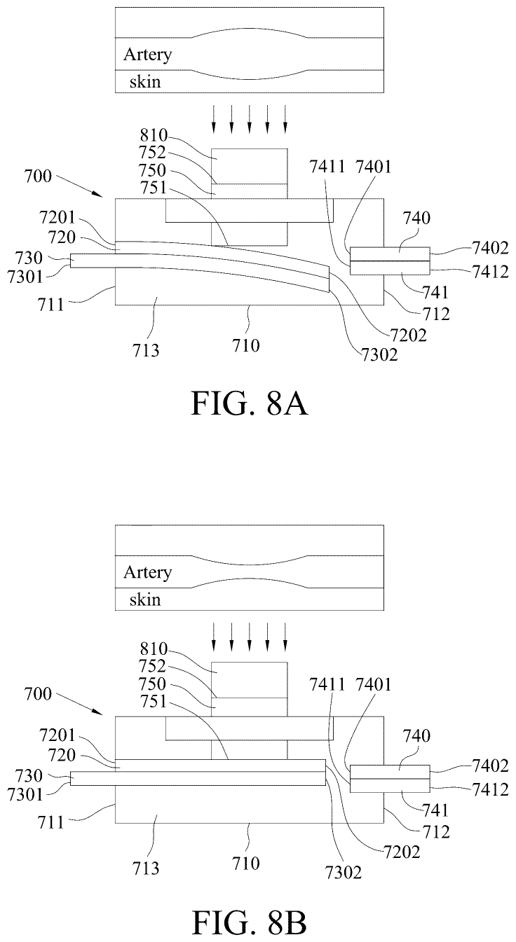

In some embodiments, a number N of the plurality of detection fibers 740 is set to two. Specifically, referring to FIGS. 8A and 8B, the second end 7302 of the input fiber 730 is placed along the middle line of the third end 7401 of detection fibers 740 and the third end 7411 of detection fibers 741 in an initial stage where the monitoring of the subject's pulse waveform is not conducted yet (i.e. the pulse waveform of the subject is absent). Further, the displacement of the cantilever 720 caused by blood pressure induced vibration will make the input fiber 730 move to different locations relative to said two detection fibers 740 and 741 (i.e. the input fiber 730 will come in contact with one of said two detection fibers 740 and 741) depend on diastole or systole of the artery. The relative displacement of the input fiber 730 is measured (hence the light coupling between the input fiber 730 and the detection fibers 740 and 741 and the resulting pulse waveform) based on the intensity coupling between the input fiber 730 and the two detection fibers 740 and 741. Further, it should be understood that the number N of the plurality of detection fibers 740 shown in FIG. 7 is not limited to two, and can be set to different numbers based on design requirements.

The aforementioned discontinuous fiber optic sensors 700 is kind of a sensor that contains only a 1-D position detector at receiving end (which means the number N of the plurality of detection fibers 740 is arranged in line).

FIG. 7A shows an embodiment of the optical sensors 20 which is embodied as discontinuous fiber optic sensors 700 with a 2-D position sensor, where like reference numerals refer to those elements described in FIG. 7, and will not further explained. And FIGS. 7B-1 and 7B-2 show an enlarged schematic diagram of the detection fibers 700 with 2-D position sensor design.

Specifically, the discontinuous fiber optic sensors 700 of FIG. 7A different from that of FIG. 7 in that the discontinuous fiber optic sensors 700 is designed in a 2-D position sensor and the number N of the plurality of detection fibers 740 is a quadrant detector. This detector can be made of either four closely bundled fiber detectors (as shown in FIG. 7A), four micro fabricated integrated waveguide detectors or an off-the-shelf micro scale quadrant position sensor. These four detectors can be configured in a quadrant configuration (as shown in FIG. 7B-1) or cross configuration (as shown in FIG. 7B-2) so that heart induced vessel vibration and human locomotion can be detected and measured based on the following equation:

$$x ∝ \frac{( {{Int}^{′}}_{a} + {{Int}^{′}}_{b} ) - ( {{Int}^{′}}_{c} + {{Int}^{′}}_{d} )}{{Int}_{a} + {Int}_{b} + {Int}_{c} + {Int}_{d}} y ∝ \frac{( {{Int}^{′}}_{a} + {{Int}^{′}}_{c} ) - ( {{Int}^{′}}_{b} + {{Int}^{′}}_{d} )}{{Int}_{a} + {Int}_{b} + {Int}_{c} + {Int}_{d}}$$

Where Int=detector's intensity before fiber displacement, Int′=detector's intensity after fiber displacement. a, b, c, d indicate the corresponding output detectors in FIG. 7B-1 or FIG. 7B-2.

The purpose of having four detector design is to allow the sensor to be able to measure any 2D motion, movement or pulse waveform instead of just 1D motion, movement of pulse waveform. Because of this 2D motion detector design, it can actually detect any bodily movement or activities along with the pulse so as to isolate these signals from the pulse waveform when combine with spectral analysis as describing hereafter.

FIGS. 8A and 8B also show a fourth embodiment of the optical sensors 20, where like reference numerals refer to those elements described in FIG. 7, and will not further explained. Specifically, the discontinuous fiber optic sensors 700 of FIGS. 8A and 8B different from that of FIG. 7 in that a contact bump 810 (e.g. a small hemispherical contact bump) is added on second side 752 of the cylindrical applicator 750 that faces the subject's skin. Said contact bump 810 may improve sensitivity of the discontinuous fiber optic sensors 700 by improving the cylindrical applicator's 750 contact with the subject's skin and the cantilever 720.

As already discussed, the discontinuous fiber optic sensors 700 (including the embodiments shown in FIGS. 7, 8A and 8B) detect the pulse waveforms by causing misalignment between the second end of the input fiber 730 and the third ends of the detection fibers 740. As clearly seen from FIG. 8A to 8B, the cylindrical applicator 750 (whether with or without the contact bump 810) is configured to react to constriction of the subject's skin during asystole (i.e. FIG. 8A) of the artery and/or expansion of the subject's skin during diastole of the artery (i.e. FIG. 8B), thereby driving the cylindrical applicator 750 to bring the input fiber 730 on the cantilever 720 to lifting or bowing position. As a result, light comes in the input fiber 730 attenuates at the light coupling between the input fiber 730 and the detection fibers 740, allowing pulse waveform of the subject to be detected.

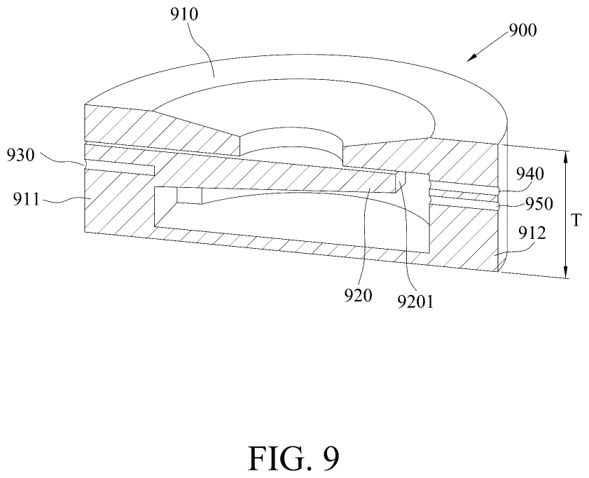

FIG. 9 shows a fifth embodiment of the optical sensors 20 (refer to as discontinuous waveguide optic sensors 900 herein) where few elements are omitted in this figure for convenience of illustrative purposes, and the features describe hereinafter such as the dimension, the shape, or the material may apply to the discontinuous fiber optic sensors show in FIGS. 6-8 as discussed above. Generally, the discontinuous waveguide optic sensors 900 described herein have a compact structure in a form of a disk (see shape of the cover 910) with the thickness T of around 2.5 mm, and the wave guide 920 has a cantilever structure with a free end 9201 (similar to the cantilever 720 in FIGS. 7-8) in a dimension of approximately 4 mm*9 mm*0.1 mm. However, the dimension and shape of the cover 910 and the wave guide 920 described herein are not meant to limit the present disclosure, and can be varied based on design requirements. In some embodiments, the free end 9201 of the wave guide 920 may move relatively to the detection fibers 940 and 950.

In some embodiments, the material of the cover 910 is preferably made of soft Polydimethylsiloxane (PDMS) through either a soft lithography processes (e.g. micro molding) or a photolithography method, and the wave guide 920 may be made of polymer materials such as Mylar, PET, PU, PMMA, or the like, through processes such as screen casting the material into a sheet, 3D printing and/or laser etching. Preferably, the wave guide is in configuration of a micro-fabricated rectangular-shaped slab or rib waveguide structure, in order to down scale of the discontinuous waveguide optic sensors 900 to microelectromechanical systems (MEMS) configuration.

In some embodiments, the input fiber 930 and the detection fibers 940 and 950 are made of core polymer optical fibers (e.g. PMMA based 240 micron core and 250 micron cladding). However, different core size of the input fiber 930 and/or the detection fiber(s) 940 may also be chosen to adjust a range or sensitivity of the discontinuous waveguide optic sensors 900 based on design requirements.

Moreover, although not illustrated, the input fiber 930 may be connected to a light source, and the detection fibers 940 and 950 may be connected to a first detector and a second detector, respectively, or they can be arranged external to the cover 910 (e.g. arranged in the transmitter 30). However, the present disclosure is not limited thereto. Further, the light source and detector(s) described herein may be configured to operate at wavelength range of, but not limited to, 880 nm.

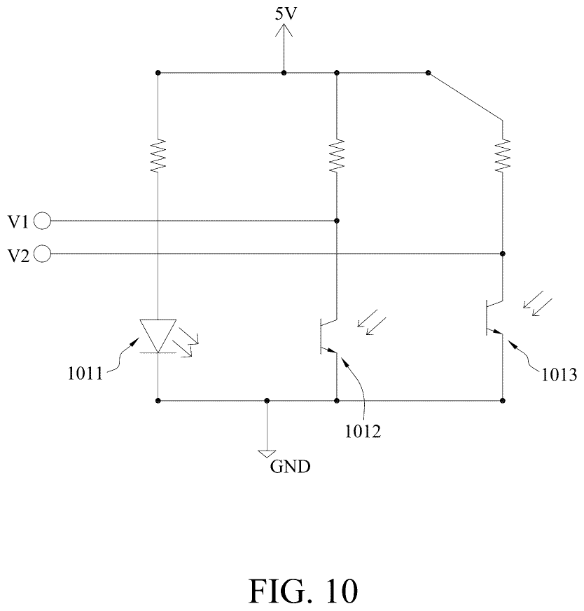

FIG. 10 illustrates an optoelectronic circuit representing general idea of how a discontinuous fiber optic sensor 900 detects pulse waveform from the subject. Particularly, the pulse waveforms can be calculated based on normalized intensity of light (produced by light source 1011, which is a LTE-4206 in this case) measured from the detection fiber(s) 940. In an embodiment where number of the detection fibers 940 is set to two and each of the detection fibers 940 are connected to respective detectors 1012 and 1013, a normalized intensity change of light in the two fiber detectors 940 of a discontinuous fiber optic sensor 900 may be expressed as the following equation:

$$\frac{{V}_{1} - {V}_{2}}{{V}_{1} + {V}_{2}} ,$$

where V₁ represents a voltage (e.g. derived from the light coupling between input fiber 930 and one of the detection fibers 940) across the detector 1012, and V₂ represents voltage (e.g. derived from the light coupling between input fiber 930 and the other one of the detection fibers 940) across the other detector 1013.

Further, the detectors 1012 and 1013 used in this optoelectronic circuit are phototransistors (e.g., LTR-4206E), which are more sensitive than other photodetectors such as photoresistors or photodiodes. However, the detectors 1012 and 1013 may be any kind of photodetectors with regard of the desired sensitivity of the overall configuration of the discontinuous fiber optic sensor 900.

It should be further noted that increased sensitivity in photodetectors comes at the price of reduced dynamic range. Dynamic range indicates the difference between the lowest and highest levels of voltage a photodetector can measure. For the optoelectronic circuit described in FIG. 10, the dynamic range of voltage capable of being measured by the detectors 1012 and 1013 is set by a bias voltage of 5V power with intensity increased from 0V, which is suitable for being processed by a 2 MHz NI DAQ system (i.e. the system adopted in the processing device 50). However, the dynamic range of the detectors 1012 and 1013 may be altered depending on types of photodetectors being used based on design requirements.

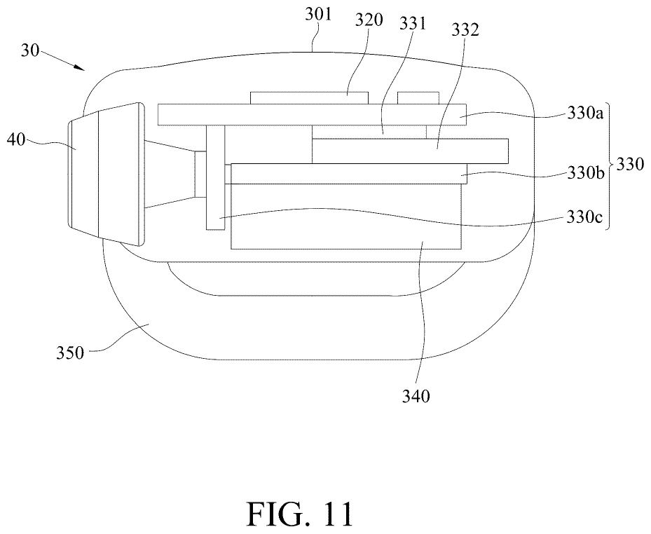

FIG. 11 shows an exemplary configuration of the transmitter 30 utilized in embodiments of this disclosure. Specifically, the transmitter 30 in this embodiment is realized as a wearable device in forms of a watch unit.

In some embodiments, a housing 301 of the transmitter 30 is injection molded in a clear plastic such as polycarbonate. The housing 301 comprises a cavity that holds all electronics (which will be further discussed hereinafter). The housing 301 also has an interface slot where the connector 40 can be accommodated. The housing 301 may has a ring structure 350 on its respective sides to hold a wristband. The housing 301 may also be further fabricated in an additive process and then post-processed for clarity.

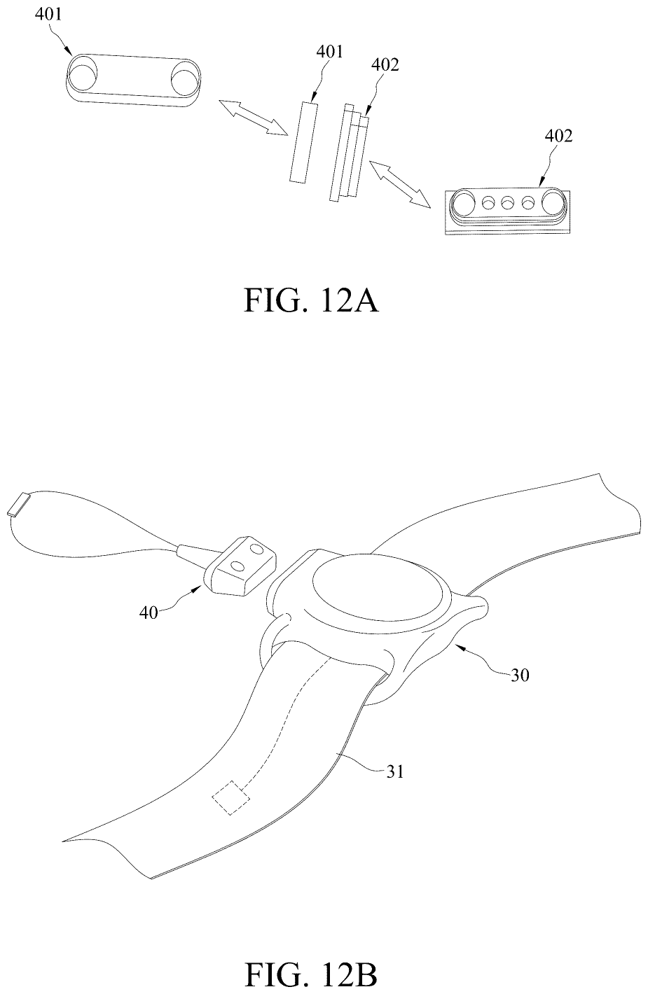

In some embodiments, the aforementioned sensor or any kinds of sensor is embedded in the wristband 31 (as shown in the FIG. 12B by the dashed rectangle). The sensing part of the sensor (e.g. the cover of the micro bend loss sensor and applicator part of the discontinuous sensor) will be strap on to the wristband 31 near the radial artery where normal blood pressure is monitored, so that there is no necessary of an additional sensor extended from the wristband. Also, the optical and electrical interconnects (represented by the dashed line running along the watch band) can also be concealed inside the wristband 31 from the housing 301.

In some embodiments, the sensor is embedded in the watch band near where the two straps come together on the wrist near radial artery. They can be strap on by Velcro based watch band and the sensor will be embedded on the side of the strap where it will come in contact with the skin.

In some embodiments, a printed circuit board (PCB) unit 330 utilized in the transmitter 30 is consist of three PCBs, namely, a micro controller unit (MCU) PCB 330a, a radio PCB 330b and an optical PCB 330c. In detail, the optical PCB 330c is designed for powering the light source for the corresponding optical sensor 20, and receiving signals (regarding pulse waveforms of a subject) from detectors for the corresponding optical sensor 20; the radio PCB 330b is designed for sending signals from said detectors to processing device 50 for further analysis; and the MCU PCB 330a is designed for managing the overall functionality of the transmitter 30. In some embodiments, the above PCBs may be integrated down to two PCBs or only one PCB based on design requirements: however, the present disclosure is not limited thereto.

In the embodiments described herein, the MCU PCB 330a is configured to carry a micro controller unit (MCU) 331 that manages the overall functionality of the transmitter 30, which comprises a 12-bit analog-to-digital converter (ADC), a plurality of digital I/O, an internal oscillator, and a capability of Universal Asynchronous Receiver/Transmitter (UART) connectivity. The MCU PCB 330 may also carry a control interface 320 for programing and testing of the functionalities of the system 10, of which may be a realization of a 7-segment display, voltage (power) regulators, headers, touch sensors, or any combination thereof.

In the embodiments described herein, the radio PCB 330b is configured to carry a Bluetooth radio 332 (such as a LMX9838SB manufactured by National Semiconductor) for sending signals from detectors to processing device 50 for further analysis, which comprises an internal voltage (power) regulator, an oscillator, and an antenna.

In the embodiments described herein, a series of resistors (not shown) may also present between interface of the MCU 331 and the Bluetooth radio 332 so as to determine a communication baud rate between said MCU 331 and Bluetooth radio 332. In addition, the MCU PCB 330a is connected to the radio PCB 330b (e.g. at the interface between the MCU 331 and the Bluetooth radio 332) by an 8-pin 0.050″ single-line connector (not shown), which is configured with flow control mechanisms such as a UART receive, transmit, ready-to-send (RTS) line, clear-to-send (CTS) line, radio status indicator, radio transmitting indicator, power and ground (GND).

In the embodiments described herein, optical PCB 330c is configured to house optical components such as light sources and detectors (not shown) for the corresponding optical sensors 20, while said light sources and detectors may be connected (e.g. via optical fibers) with corresponding optical sensors 20 through the connector 40. In addition, the optical PCB 330c is connected with the MCU PCB 330a at a right angle via a 4-pin 0.050″ single-line connector (not shown), which is configured with flow control mechanisms such as power, GND and signal transceivers for detectors. Furthermore, the optical board 330c also carries surface mount resistors (not shown) to provide sufficient voltage (power) to the light sources, and signal resistors to the detectors for receiving signals regarding pulse waveforms of a subject. Moreover, the optical PCB 330c may optionally include pads for capacitors for passive filter implementation.

In some embodiment, each PCBs 330a, 330b and 330c of the transmitter 30 may include its own voltage regulator (in addition to the voltage regulators in the MCU PCB 330a) to limit the effect of switching components on the optical PCB 330c and the radio PCB 330b.

In the embodiments described herein, a battery 340 utilized in the transmitter 30 is a lithium ion polymer battery (e.g. a GM482030 manufactured by Powerstream) to power the transmitter 30. Lithium ion polymer batteries acquires advantages such as high energy density and stability, and generally includes an internal protection circuit that prevents overcharge, overdischarge, and short circuit damage. However, the choice of battery 340 is not meant to restrict the scope of this disclosure, and can be varied based on design requirements.

Overall, the transmitter 30 described above may act as a base station for various optical sensors 20 to be interfaced, such that signals regarding pulse waveforms from different body parts of a subject may be monitored by various optical sensors 20 simultaneously and delivered to the processing device 50 therefrom.

FIGS. 12A and 12B shows the assembly and mechanism of how a connector 40 is provided to connect the optical sensors 20 with the transmitter 30.

As seen from FIG. 12A, the connector 40 comprises a male part 401 on a sensor side for receiving optical fibers (e.g. the optical fiber 230, the input fibers 630, 730, and 930, the detection fibers 640, 740, and 940, etc.) from the optical sensors 20 and a female part 402 on an transmitter side for connecting the optical fibers to the corresponding light sources and detectors resided within the transmitter 30 (as explained in FIG. 11), hence secure simple light coupling between said light sources, optical fibers and the detectors. Both the male part 401 and the female part 402 of the connector 40 may be formed through hot embossed or hot molded method.

In the embodiments described herein, each of the male part 401 and the female part 402 comprises a set of same numbers of magnets (e.g. neodymium magnets), respectively. Referring to the two large through holes (i.e. the location of said magnets) on each of the male part 401 and female part 402 illustrated in FIG. 12A, the location of the magnets are designed correspondingly to provide easy self-aligned coupling when male part 401 is to be coupled with the female part 402. It should also be noted that the numbers of magnets in the male part 401 and the female part 402 may be varied based on design requirements, and the present disclosure is not limited thereto.

Referring to FIG. 12B, when the male part 401 and the female part 402 are coupled together, the connector 40 has a tapered shape to allow easy insertion into the interface slot on the housing 301 of the transmitter 30. In this case, the magnets on each of the male part 401 and the female part 402 should be aligned in place to create reliable electrical connection, so as to allow current from battery 340 of the transmitter 30 pass from the optical PCB 330c to the light sources through the magnets. In some embodiments, the transmitter 30 is further configured to not power on unless all magnets on the male part 401 and female part 402 are aligned in place, so as to ensure efficient battery 340 usage and proper optical alignment within the sensors 20, light sources and the detectors.

In some embodiments described herein, with the discontinuous fiber sensor design, behind the middle opening of the female part 402 is a LED which provides the light to input fiber, and the two neighboring openings (neighbor to the middle opening) house two separate photodiodes connected to two detector fibers.

In some embodiments described herein, a strain relief feature (referring to the three smaller through holes shown on female part 402) is also added to the connector 40 to protect the optical fibers from excessive bending.

Referring back to FIG. 1, the processing device 50 is implanted with a data acquisition (DAQ) system, which is configured to perform tasks such as data acquisition of signals regarding the subject's pulse waveform and blood pressures, processing the signal, analyzing the signal, display pulse waveform and blood pressures, or providing user interface for viewing options. A system-design platform (e.g. NI LabVIEW) may be utilized to develop such DAQ system. More specifically, the DAQ system adopted by the processing device 50 should be developed to operate at appropriate relatively high speed to ensure high enough sampling rate and resolution to capture all the fast and slow moving pulse waveforms of the subject such as forward propagating waves and reflecting pressure waves. In addition, techniques of artificial intelligence and big-data may also be introduced in the DAQ system to cope with large amount of data obtained from each subject to improve quality and precision of data analysis.

Through the design of the DAQ system, the processing device 50 may display a graphical user interface (GUI) through its display that provides options for choosing a subject (patient) of interest, reviewing a session or starting a new session for pulse waveform monitoring of a subject, configuring individual measurements for selected subject, calibration, and/or displaying a real-time measurement regarding pulse waveforms and blood pressures of the subject.

In preferred embodiments described herein, the GUI provided by the DAQ system is configured to graphically present pulse waveform and blood pressure of a subject in a real-time measurement window on a top pane of the display, present a navigation window on left pane of the display to allow users to shift easily from one window to another based on selection of a menu therefrom, and present a summary window on bottom pane of the display which summarizes calculations for each measurement of the pulse waveform and blood pressures of a subject.

Further, unwanted body motions and activities often affect the accuracy of pressure and heart rate measurement. In particular human periodic motion, Fourier transforms can be used to detect or recognize these periodicities. In the proposal, a motion-based frequency-domain analysis is implemented for both heart rate and human periodic motion recognition. Human motion analysis is the systematic study of human motion by careful observation, augmented by instrumentation for measuring body movements, body mechanics and the activity of the muscles. It aims to gather quantitative information about the mechanics of the musculoskeletal system during the execution of a motor task.

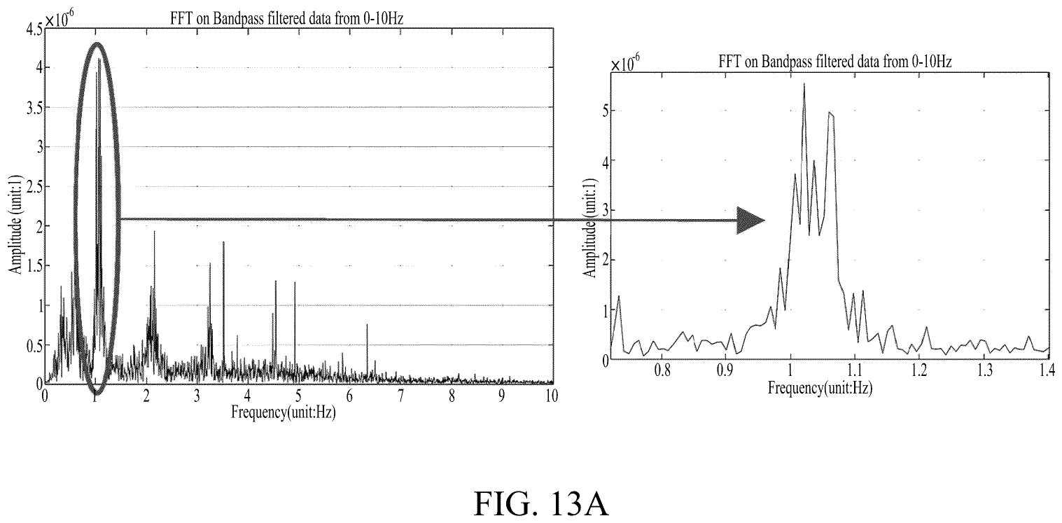

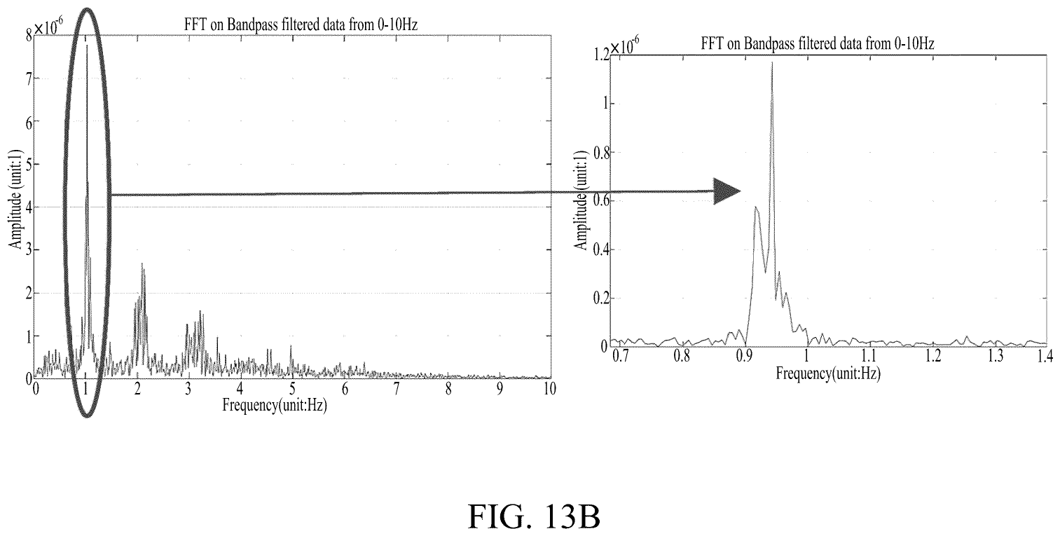

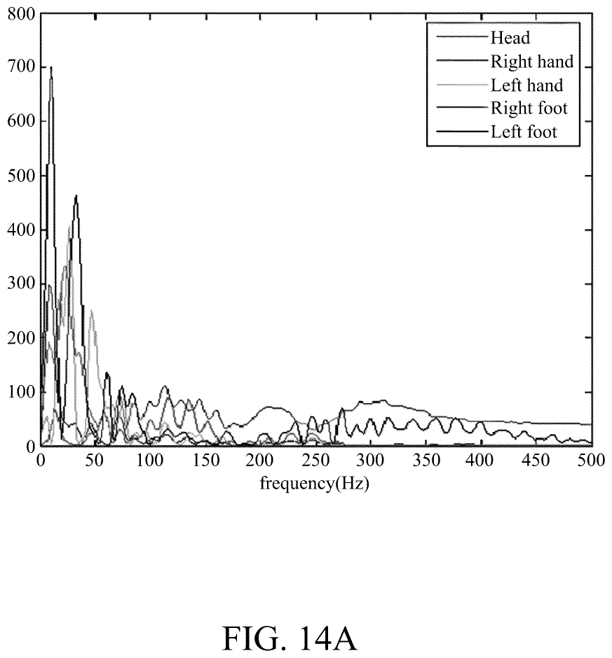

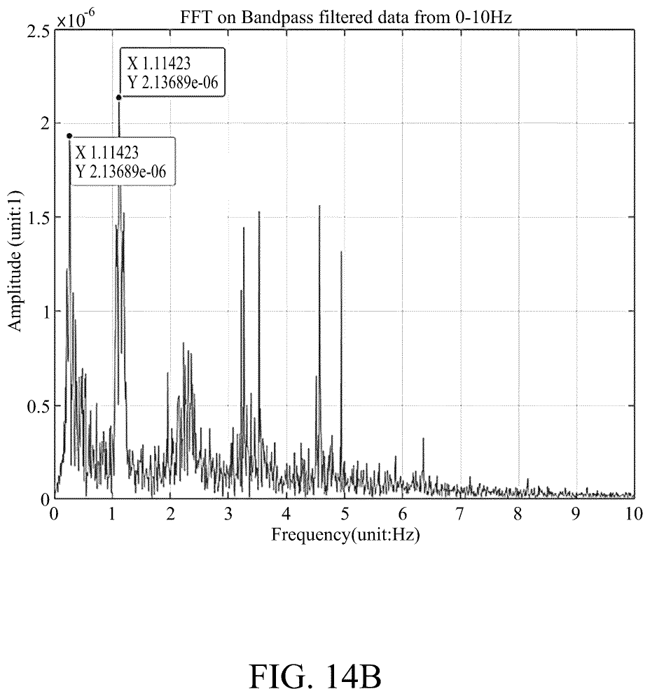

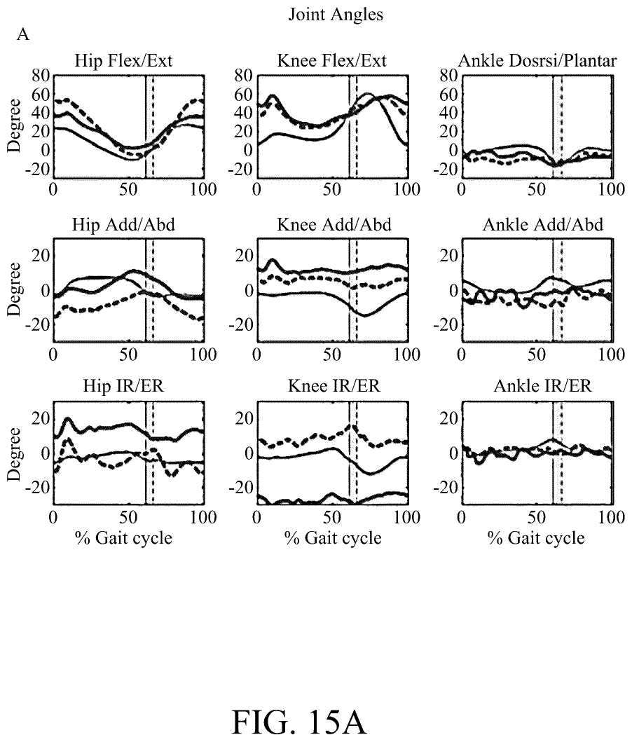

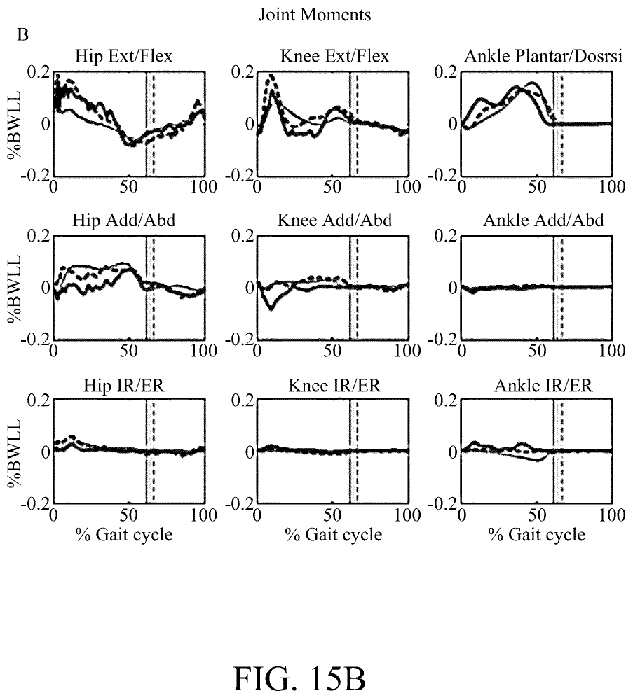

However, in calibrating the system, these motions will be carefully studied and analyzed along with the spectral measurement thus they can be recognized and filtered from the heart waveform measurement. Further, human activity frequencies are between 0 and 20 Hz, and that 98% of the FFT amplitude is contained below 10 Hz (as shown in FIG. 13, FIG. 14 and FIG. 15). Because of these signals can be detected and distinguished from the pulse waveform in the frequency spectrum, they can be filtered. Hence, the overall blood pressure waveform measurement improves.

FIG. 13A shows the corresponding X displacement spectrum form the quadrant sensor, and FIG. 13B shows the corresponding Y displacement spectrum from the quadrant sensor. In addition, patient's breathing, hand, arm and knee movement, or subject engaging in walking or running can be clearly identified and amplitude measured based on the quad sensor's xy position spectral measurement (respectively shown in FIG. 14B and FIG. 14A). The recognition results demonstrate that feature-based spectral analysis allows classification of periodic motions from low-level, unstructured interpretation without recovering underlying kinematics. This motion-based frequency-domain method avoids a time-consuming recovery of underlying kinematic structures in visual analysis while obtaining more accurate blood pressure waveform measurement. Further, FIG. 15A and FIG. 15B show examples of knee bending as a function of frequencies with joint angles and joint moments respectively.

Therefore, the present disclosure further provides a motion-based frequency-domain method for monitoring pulse waveform and blood pressure of a subject, comprising: providing the optical sensor of the present disclosure: measuring the pulse waveform and blood pressure of the subject by the optical sensor to generate a pulse waveform and blood pressure signal: recognizing motion signal generated by the subject: filtering the motion signal out from the pulse waveform and blood pressure signal; and reconstructing an accurate pulse waveform and blood pressure signal. The motion-based frequency-domain method avoids a time-consuming recovery of underlying kinematic structures in visual analysis while obtaining more accurate blood pressure waveform measurement.

In some embodiments, the motion signal is generated by the motion such as subject's breathing, hand, arm and knee movement, or the subject engaging in walking or running.

In the optical sensor and a system for monitoring pulse waveform and blood pressure of a subject as described in the present disclosure, optical sensor may be manufactured with compact structure, low profile, low cost, and exhibits benefits of disposability, easy to apply, immunity to electro-magnetic interference, high sensitivity, having minimal affect towards sense of touch, maintains patient safety, and supportive of accurate real-time measurements for the clinician. Therefore, pulse waveform and blood pressures of a subject may be faithfully monitored continuously throughout day and night, so as to provide abundant prognostic information while avoiding interference in normal daily activity of the subject.

The present disclosure has been described with exemplary embodiments to illustrate the principles, features, and efficacies of the present disclosure, but not intend to limit the implementation scope of the present disclosure. The present disclosure without departing from the spirit and scope of the premise can make various changes and modifications by a person skilled in the art. However, any equivalent change and modification accomplished according to the disclosure of the present disclosure should be considered as being covered in the scope of the present disclosure. The scope of the disclosure should be defined by the appended claims.

1. An optical sensor for monitoring pulse waveform and blood pressure of a subject, comprising:
a cover having a chamber formed therein, wherein an outer surface of the cover has an input port and an output port formed thereon;
an input waveguide having a first end and a second end and configured to guide a light from the first end to the second end, wherein the second end of the input fiber is disposed in the chamber through the input port; and
a first detection waveguide having a third end and a fourth end and configured to guide the light from the third end to the fourth end, wherein the third end is disposed in the chamber through the output port and configured to receive the light transmitted from the second end of the input waveguide;
wherein the second end of the input waveguide is not in contact with the third end of the first detection waveguide, and the second end of the input waveguide is configured to move relatively to the third end of the first detection waveguide.

2. The optical sensor of claim 1, further comprising:
a second detection waveguide having a fifth end and a sixth end and configured to guide the light from the fifth end to the sixth end, wherein the fifth end is disposed in the chamber through the output port and configured to receive the light transmitted from the second end of the input waveguide;
wherein the second end of the input waveguide is not in contact with the fifth end of the second detection waveguide, and the second end of the input waveguide is configured to move relatively to the fifth end of the second detection waveguide; and
wherein the third end of the first detection waveguide is adjacent and parallel to the fifth end of the second detection waveguide, and the second end of the input waveguide is disposed along the middle line of the third end of the first detection waveguide and the fifth end of the first detection waveguide.

3. The optical sensor of claim 1, wherein the first detection waveguide comprises four micro waveguides forming a quadrant detector configured in a quadrant configuration or a cross configuration.

4. The optical sensor of claim 1, wherein the chamber is sealed with a soft membrane.

5. The optical sensor of claim 1, further comprising:
a plastic deforming plate having a free end coupled to the second end of the input waveguide in the chamber.

6. The optical sensor of claim 5, further comprising:
a cylindrical applicator penetrating through the cover, wherein the cylindrical applicator has a first side in contact with the cantilever and a second side opposite the first side.

7. The optical sensor of claim 6, further comprising a bump disposed on the second side of the cylindrical applicator.

8. The optical sensor of claim 1, wherein the first end of the input waveguide is configured to couple with a light source directly or by a first optical fiber, the fourth end of the first detection waveguide is configured to couple with a detector directly or by a second optical fiber.

9. An optical sensor for monitoring pulse waveform and blood pressure of a subject, comprising:
a first plate having a first engaging side;
a second plate having a second engaging side facing the first engaging side;
an optical fiber sandwiched between the first engaging side of the first plate and the second engaging side of the second plate, wherein the first engaging side and the second engaging side have corrugated teeth structure formed thereon for engaging the optical fiber.

10. The optical sensor of claim 9, wherein the corrugated teeth are spaced from each other with a pitch, and the optical fiber exhibits periodic bending at the pitch of the corrugated teeth; and wherein the optical fiber is configured to guide a light from a light source to a detector, and attenuation of the light in the optical fiber is proportional to amount of the periodic bending of the optical fiber.

11. The optical sensor of claim 10, wherein a signal regarding the pulse waveform and blood pressure of the subject is produced by the detector by detecting a light loss during the attenuation of the light related to a force applied to the optical sensor.

12. A system for monitoring pulse waveform and blood pressure of a subject, comprising:
a transmitter comprising a light source and a detector, and the transmitter is configured to collect and deliver signal regarding the pulse waveform produced by the detector;
the optical sensor of claim 1, coupled to the transmitter by a connector and configured to guide a light from the light source to the detector, and
a processing device, configured to process the signal delivered by the transmitter.

13. The system of claim 12, wherein the transmitter further comprises:
a housing having an interface slot for accommodating the connector.

14. The system of claim 13, wherein the connecter has a taper structure for allowing insertion into the interface slot or relieving strain of the input waveguide or the first detection waveguide.

15. The system of claim 12, wherein the transmitter further comprises:
an optical printed circuit board disposed in the housing and configured for carrying the light source and the detector, powering the light source, and/or receiving the signal from the detector;
a radio printed circuit board disposed in the housing and configured for carrying a Bluetooth radio and/or sending the signal to the processing device; and
a printed circuit board carrying a micro controller unit for managing functionality of the transmitter.

16. The system of claim 15, wherein the printed circuit board carrying the micro controller further carries a control interface for programing and testing of functionality of the transmitter, and wherein the control interface comprises a 7-segment display, a voltage regulator, a header, a touch sensor, or any combination thereof.

17. The system of claim 12, wherein the connector comprises:
a male part for receiving the input waveguide and the first detection waveguide; and
a female part for connecting the input waveguide and the first detection waveguide to the transmitter in accordance with disposition of the light source and the detector,
wherein the male part is couple to the female part.

18. The system of claim 17, wherein the male part is magnetically coupled to the female part.

19. The system of claim 18, wherein the male part has a first set of magnets, and the female part has a second set of magnets, wherein the first set of magnets and the second set of magnets corresponds in numbers and location to allow self-aligned coupling between the male part and the female part.

20. The system of claim 19, wherein the transmitter is configured to not power on unless the first set of magnets and the second set of magnets are aligned in place.

21. The patch sensor system of claim 12, wherein the processing device is configured with:
a data acquisition (DAQ) system for analyzing the signal delivered by the transmitter; and
a graphical user interface (GUI) for displaying the pulse waveform and blood pressure according to the signal.

22. The patch sensor system of claim 12, the transmitter further comprising a wristband, wherein the housing has a ring structure configured to hold a wristband, and the optical sensor is embedded in the wristband near a radial artery of the subject.

23. A method for monitoring pulse waveform and blood pressure of a subject, comprising:
providing the optical sensor of claim 3;
measuring the pulse waveform and blood pressure of the subject by the optical sensor to generate a pulse waveform and blood pressure signal;
recognizing motion signal generated by the subject;
filtering the motion signal from the pulse waveform and blood pressure signal; and
reconstructing an accurate pulse waveform and blood pressure signal.
